(19) United States
(12) Patent Application Publication
(10) Pub. No.: US 2017/0209075 A1
(43) Pub. Date: Jul. 27, 2017

(54) MEASURING DEVICE AND SYSTEM FOR COMPREHENSIVE SCREENING FOR CHILDREN AND ADOLESCENTS

(71) Applicant: 3DMD sp. Z o.o., Rzeszow (PL)

(72) Inventor: Marcin LEWANDOWSKI, Warszawa (PL)

(21) Appl. No.: 14/900,127

(22) PCT Filed: Oct. 5, 2015
(86) PCT No.: PCT/IB15/57611

§ 371 Date: Dec. 18, 2015

(30) Foreign Application Priority Data
Jul. 16, 2015 (PL) PL413147

(51) Int. Cl. A61B 5/107; A61B 5/024; G01N 27/02; A61B 5/145; G01G 19/50; A61B 5/053; A61B 5/021

(52) U.S. Cl. CPC A61B 5/1072; A61B 5/0537; A61B 5/1074; A61B 5/024; A61B 5/021; A61B 5/14532; G01G 19/50; G01N 27/02

(57) ABSTRACT

The measuring device for comprehensive screening for children and adolescents includes a base and a body in the form of a component vertically ending at the top with an arm mounted at the angle of 90°, rotationally in the horizontal plane, positioned on the base. On the arm, there are a motion sensor system and a height measurement system. Additionally, on the vertical component, there is at least one interactive electronic device, a bioelectrical impedance (BI) measurement system, and a waist and hip circumference measurement system. The base includes a built-in body weight measurement system. The object of the invention is also a measuring system.

### CROSS-REFERENCE TO RELATED APPLICATIONS

See also Application Data Sheet.

### STATEMENT REGARDING FEDERALLY SPONSORED RESEARCH OR DEVELOPMENT

Not applicable.

### THE NAMES OF PARTIES TO A JOINT RESEARCH AGREEMENT

Not applicable.

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not applicable.

### STATEMENT REGARDING PRIOR DISCLOSURES BY THE INVENTOR OR A JOINT INVENTOR

Not applicable.

### BACKGROUND OF THE INVENTION

1. Field of the Invention

The object of the invention is a functional device for comprehensive screening for children and adolescents enabling carrying out comprehensive tests of primary vital indicators and anthropometric parameters such as height, weight, length and corpulence of foot, and waist and hip circumference, and arterial blood pressure, body fat, heart rate, as well as minimally invasive measurements of parameters, e.g, glucose level. The device has the form of a stylized giraffe, which allows to minimize the problem of children's reluctance towards medical appointments and examinations. The object of the invention is also a measurement system.

2. Description of Related Art Including Information Disclosed Under 37 CFR 1.97 and 37 CFR 1.98

The prior art has many devices concerning measurements of primary vital indicators and anthropometric measurements. Among the devices that concern the measurement of one parameter there can be distinguished those that enable the measurement of the child's height alone in different ways.

There are many devices enabling measuring the distance between the sensor and the object which use different measurement techniques. Among the available measuring devices there can be distinguished:

a) ultrasonic sensors to measure the distance

b) laser sensors to measure the distance

c) devices measuring the height of objects based on the analysis of scanned images (checkpoints)

It is advisable to develop a device that uses an ultrasonic sensor of distance with a measurement parameters resolution reaching 1 mm. The feature of such a device is dictated by the advantages of the technology based on the measurement of ultrasound that is safe for the measured person and at the same lime gives very good measurement results.

The patent application PL355926 relates to a method and a device for documenting the child's height. On the other hand, the invention CN103099621A discloses a child height ruler. The child height ruler comprises a height ruler body and a mark that can be bound to it.

The utility model CN2719227 defines an automatic measuring and analyzing system of children's physique height, comprising a measuring part, a display part and a storing pail. The utility model is characterized in that the automatic measuring and analyzing system of children's physique height is composed of an ultrasonic sensor, a weighting sensor, an A/D converter with an analog switch, an 89C52 single chip computer, a USB communication module and a computer.

A solution designed to take measurements of body fat is also known.

The object of the invention PL194850 is a skinfold caliper for measuring skinfolds. Measurements of skinfolds enable to determine the content of fat tissue and its distribution in the body, as well as to assess nutrition. On the other hand, the object of the patent application PL403544 is a method for reducing body fat, particularly in certain parts of the body of an overweight or obese subject. It is characterized in that the food intolerance test is carried out in a subject, then the content of fat in the body of the subject is determined using bioelectrical impedance analysis (BIA), subsequently the anthropometric measurements are performed, and a diet for the subject is determined. Then procedures are carried out for certain parts of the subject's body to assist the reduction of local fat accumulation while maintaining firmness of the skin.

The invention CN2179102Y discloses a human body subcutaneous fat measuring device characterized in that the measuring device Is composed of a left tong ram with a force-adding arm and a scale plate, and the right tong ram of upper and lower baffle plates which are matched with the scale plate in a sliding mode. The human body subcutaneous fat measuring device has simple structure and convenient operation, and is used for monitoring the nutrition situation of 0-6 year-old children and self health care.

Among the solutions indicating the ways to measure posture, for example, the invention PL065381 draws attention, which reveals a station for measuring the posture, especially of children, designed for preventive screening in educational institutions, outpatient and rehabilitation clinics, as well as corrective gymnastic workplaces. The station for measuring the posture is composed of a podoscope, on the upper surface of which there are arranged hoards with a posturographic grid printed. Opposite to the board there is an electronic image recorder, set on a tripod, connected to the computer having a printer. The podoscope of the station is composed of a base having glass on its upper surface, under which there is a mirror set at an angle which transfers the image of the feet from the bottom to the image recorder, which is connected to the computer or the station.

The second group of measuring devices includes those that are designed for comprehensive measurements of a greater number of vital indicators. The invention PL217840 is a device for measuring vital signs used for simultaneous measurement of heart rate, respiratory rate and temperature, based on an analysis of the optical signal equipped with coded sensors.

On the other hand, the invention PL166630 is a device for measuring the anthropometric parameters of posture of the human body, characterized in that the measuring head, on which two measuring crossheads arranged in mutually perpendicular directions are positioned, is mounted on the end of an assembly of straight-line mechanisms connected in a movable way to a rotary head mounted on a vertical column. The rotary head is equipped with three measuring components driven by the movements of straight-line mechanisms in three mutually perpendicular directions.

The patent application PL383129 defines a method and a device, wherein the sensor of vertical slide measures the vertical displacement of the measuring crosshead. The sensor of the interposition of the measuring crosshead measures the horizontal displacement of the measuring crosshead. The sensor of the rotation measures the angular displacement of the measuring crosshead. The measured displacement values are processed into electrical signals, and their values are counted by the counters of the vertical, horizontal and angular displacement, wherein each measurement starts from zeroing counter displays of all sensors. After measuring, a three-dimensional image of person is created, on the basis of which the anthropometric parameters of the human body are read. The device has a measuring head, rotatably and slidably mounted on a tripod of the anthropometer, using a vertical displacement sleeve, through two pressure bearings.

Another solution in the form of the utility model PL056039 discloses a kit for measuring blood pressure, electrocardiography of the heart, urinalysis, measuring blood sugar, and other medical devices.

The invention CN2868184Y relates to a wireless health monitoring system and is an information system for sending physiological parameters e.g. cardiogram, pulse rate, blood pressure and temperature, etc.) in the elderly, the sick, children and other monitored individuals through wireless technology for collecting, monitoring and analyzing the data in real time.

The utility model JP2002045343A describes an invention in the form of sensors which can be easily operated by the elderly and children and is convenient for them for the daily measurements, which measure their health condition. The solution consists in the fact that the sensorcomprise a body imitating an animal. Cardioelectrodes are positioned in both ears of the animal, namely in a pair of symmetrical hanging ears, which are allowed to contact when grasped with fingers when performing the electrocardiography. The temperature sensor is located in the projecting portion of the tail and the body temperature is measured while the patient grasps the temperature sensor from the sensor-housing component and puts it under their arm. The SPO2 sensor is positioned in the concavity of the left arm and the SPO2 is performing measurements when the patient is placing the finger in it. This invention, however, is not used to measure a number of other vital and anthropomorphic parameters like the device being the subject of the present invention.

On the other hand, the invention JP2003135405 discloses medical equipment designed in the form of a rabbit. This solution gives the results of measurements and therapeutic effects. The head is equipped with a control section display. The display is operated by a touch panel. The control and display section has a display area for displaying measurement data and displaying image animations. The maximum value of blood pressure and the minimum value of pressure are displayed in the measurement data window. Moreover, ears and tail are moving.

In the absence of a device enabling comprehensive monitoring, diagnosing and consequently taking preventive measures in terms of early detection of obesity, underweight, arterial hypertension in children and adolescents, it is desirable to develop such a device that will comprehensively monitor primary vital parameters and anthropometric indicators.

### BRIEF SUMMARY OF THE INVENTION

The device being the subject of the invention is the result of industrial work aimed at creation of a device to support the work of doctors and to enable common access of the youngest patients to primary examinations. The benefits resulting from the device being the subject of the present invention are very valuable for healthcare providers, educational institutions, commercial customers and the subjects themselves. The device will be a link in a campaign to prevent overweight and underweight, building awareness of the importance of taking care of health even in the youngest children, children in school age and adolescents. The device has a touch screen that will allow for entering date of birth of the child, the results of examinations, and e-mail addresses or phone numbers, thanks to which it will be possible to send the results of examinations to the parent or the attending physician for archiving.

The device was created in a child-friendly form showing the animal in the form of a giraffe, which minimizes the problem of children's reluctance towards medical appointments or examinations. The individual measurements are carried out by certain animals, and the results of these examinations will be compared to the height or weight of the animals in the world (the educational function of the device).

The essence of the invention is a measuring device for comprehensive screening for children and adolescents comprising a body in the form of a component vertically ending at the top with an arm mounted at the angle of 90°, rotationally in the horizontal plane, positioned on the base and characterized in that, on the arm, there are a motion sensor system and a height measurement system. Additionally on the vertical component, there is at least one interactive electronic device, a bioelectrical impedance (BI) measurement system, a waist and hip circumference measurement system, while the base comprises a built-in body weight measurement system.

Preferably, the measuring device comprises a motion sensor system, comprising a motion sensor, a microcontroller and a Bluetooth module.

Preferably, the height measurement system consists of an ultrasonic distance sensor, a microcontroller and a Blue tooth module.

Preferably, the interactive electronic device is an interactive tablet with a built-in communication modem.

Preferably, the bioelectrical impedance (BI) measurement system consists of an electrode, an ADC converter, a DAC converter, an electronic system for the BI measurement, a microcontroller, and a Bluetooth module.

Preferably, the waist and hip circumference measurement device is in the form of a hoop or a belt or a measuring tape with a centimeter and inch scale, as well as the system measuring the level of projection of the measuring tape.

Preferably, the body weight measurement system consists of a pressure sensor load, an ADC converter, a microcontroller, and a Bluetooth module.

Preferably, the measuring device additionally comprises an interactive electronic device in the form of an informational tablet.

Preferably, the measuring device additionally comprises the pulse rate measurement system consisting of a diode sensor, a microcontroller and a Bluetooth module.

Preferably, the measuring device additionally comprises the arterial blood pressure measurement system, consisting of a sleeve for analog measurement of changes in blood pressure, an amplifier with an ADC converter, a microcontroller, and a Bluetooth module.

Preferably, the measuring device comprises software.

Preferably, the measuring device comprises a Bluetooth module for communication of peripheral devices with a microcontroller in order to read the measurement carried out.

Preferably, the measuring device comprises a server in the form of a computer collecting, processing and storing the collected data.

Another object of the invention is a measurement system for comprehensive screening for children and adolescents, comprising the above-described measuring device and a server, connected one to another via a communication network, characterized in that the measuring device performs the measurement of the given parameter after their initiation by the user, stores the result and then sends the result to the server where the results are saved, processed and stored.

Preferably, the system is characterized in that the server comprises a communication module.

The device being the subject of the invention increases health self-awareness in children. It is an innovative solution that responds to the increasing trend towards personalization of the approach to treating the patient—not only in terms of treating each patient individually, but also allowing the patient self-observation and mobile cooperation with a physician.

With the device, a parent may at an time carry out examinations of primary indicators, conduct their observation while engaging the child in it and inform a physician about them by email or by SMS text message.

Ultimately, the device may constitute equipment of schools, kindergartens, offices of general practitioners and pediatricians, as well as hospitals and pediatric wards. The device can also be placed in supermarkets, stores with products for children, swimming pools and playrooms. It can also be equipment in medical waiting rooms.

The device allows functioning on various global markets, including in particular the following markets: European—North American and Russian.

### BRIEF DESCRIPTION OF THE SEVERAL VIEWS OF THE DRAWINGS

The invention has been further described in the embodiment and on the drawings.

FIG. 1 shows a schematic view of the measuring device.

FIG. 2 shows a schematic view of the measuring device with additionally mounted systems for measuring arterial blood pressure and pulse rate.

FIG. 3 shows a schematic illustration of a diagram of the body weight measurement system.

FIG. 4 shows a schematic illustration of a diagram of the bioelectrical impedance (BI) measurement system.

FIG. 5 shows a schematic illustration of a diagram of the system for measuring the pulse rate in the examined person.

FIG. 6 shows a schematic illustration of a diagram of the arterial blood pressure measurement system.

FIG. 7 shows a schematic illustration of the architecture of the measuring device.

FIG. 8 shows a schematic illustration of the architecture of the measuring device with additional arterial blood pressure and pulse rate measurement systems.

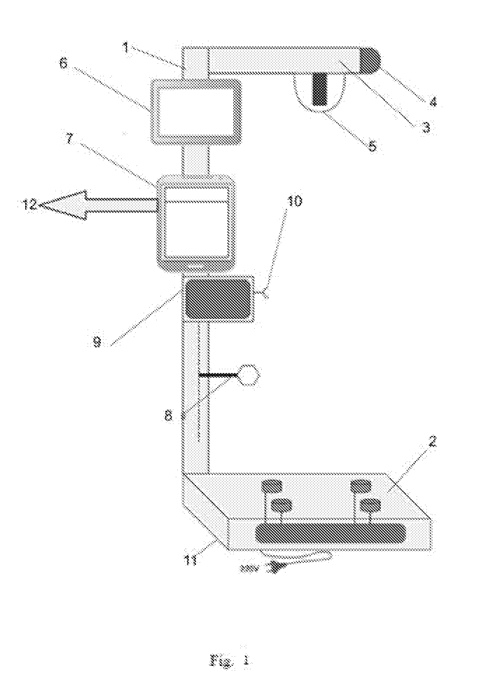

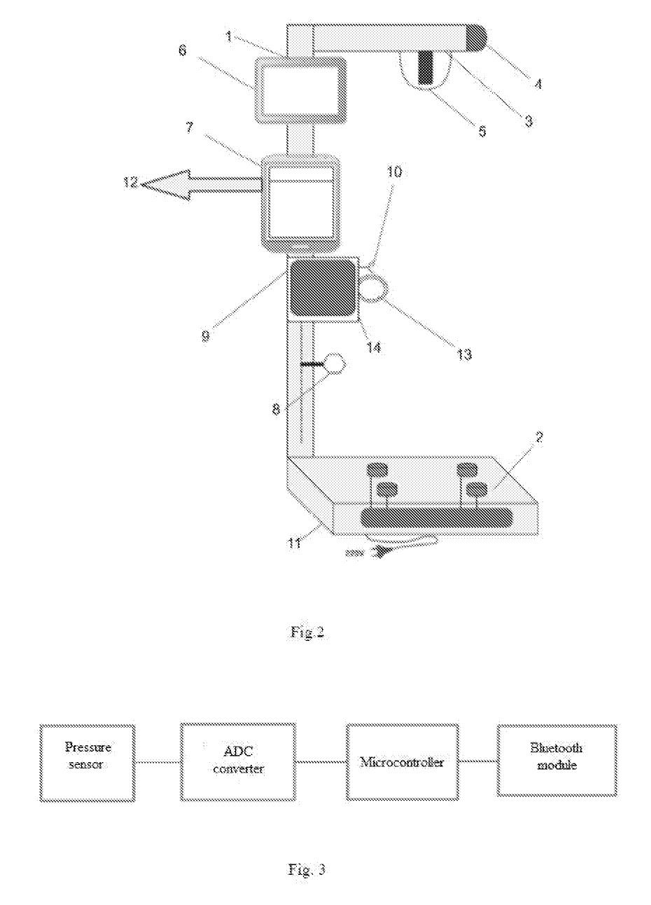

### DETAILED DESCRIPTION OF THE INVENTION

The measuring device for comprehensive screening for children and adolescents being the object of the invention consists of three main component groups:

a) body—the main unit, created in a child-friendly form, which minimizes the problem of children's reluctance towards medical appointments or examinations; the body was designed in the form of a vertical element 1 with an arm 2 and a base 3, which has a built-in scale; the vertical element 1 is made on the outside of transparent materials, durable with smooth surfaces and illuminated from the inside, which allows the outer surfaces of the body (a totem pole) to be illuminated from the inside—with a backlight—and thus the whole device will have an attractive form not only in well-lit, but also in less-lit places; the whole body on the outside will resemble an animal in the form of a giraffe, thanks to colorful graphics of the whole device;

b) peripheral devices—devices for comprehensive screening measurements of primary vital indicators and anthropometric parameters (e.g. aimed at measuring the child's height, body fat, arterial blood pressure and pulse rate;

c) software in web and mobile versions—that is, among others, a mobile application for entering data on the tablet.

The body of the device is a housing for mounting peripheral devices (tablets, measurement systems, belt for measuring waist and hip circumference) designed and constructed in a way that is aesthetic and attractive for children. The body allows installation of informational and technical equipment inside the housing.

Peripheral devices occurring in the device being the object of the invention can be divided into two groups: the devices that are accessible from the outside by the user and the internal devices constituting the information and technical architecture of the device.

The first group of devices, accessible by the users of the device and used by them to carry out a comprehensive screening of primary vital indicators and anthropometric parameters, as well as measurement of arterial blood pressure, body fat and heart rate, includes:

a) informational tablet 6;

b) interactive tablet 7;

c) height measurement system 5;

d) BI measurement system 9 with electrodes to measure BI 10;

e) arterial blood pressure measurement system 13;

f) waist circumference measurement device 8;

g) motion sensor system 4;

h) pulse measurement system 14;

i) body weight measurement system 11;. And

j) server 12.

The informational tablet 6 was used for the presentation of animation encouraging children to use the device, information on the operation of the whole device, information encouraging to interact with the user in order to perform measurements by the device, educational content related to healthy nutrition and a comprehensive screening in children. The task of the interactive tablet 7 (with 3G modem) for data entry and collection of data from the microcontroller is to obtain data from measurement systems (measurement of height, measurement of length and corpulence of foot, BI, body weight, as well as pulse rate and arterial blood pressure in an extended version of the device) and from the user—the data entered in the information form. Another function is data transfer to the server using the built-in 3G modem.

The height measurement system 5 is an electronic device that allows measurement of the examined person's height and providing the measurements carried out is Bluetooth. The measurement system consists of the following components:

a) ultrasonic distance sensor;

b) microcontroller;

c) Bluetooth module.

With the help of the height measurement system 5 it is also possible to measure the length and corpulence of foot.

With the body weight measurement system 11 consists of the pressure sensor, the ADC converter, the microcontroller and the Bluetooth module as shown in FIG. 3.

The pressure sensor is a device that converts the change in a pressure force to the change in an output voltage of the sensor. The ADC converter converts an analog signal from the pressure sensor into a digital signal. A digital body weight measurement is carried out in the microcontroller. The Bluetooth module is used for communication of peripheral devices with the microcontroller in order to read the measurement performed.

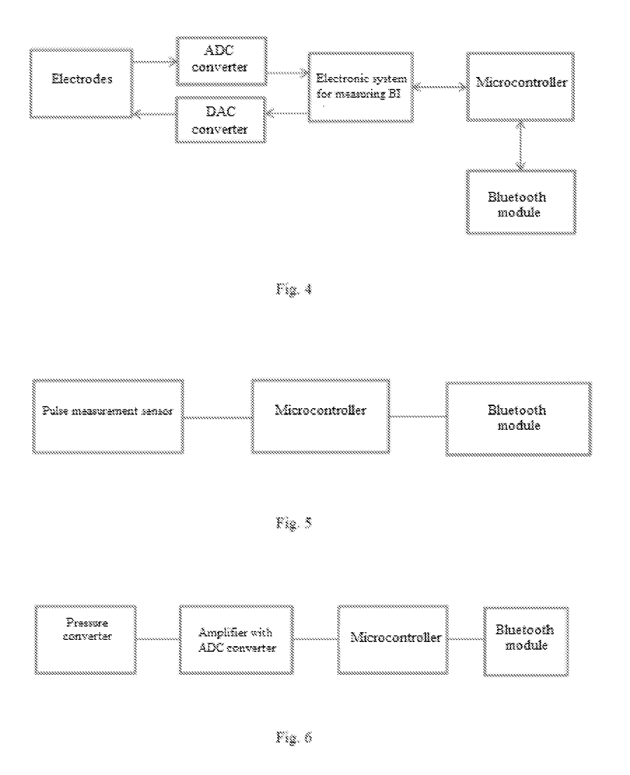

The bioelectrical impedance (BI) measurement system 9 consists of the electrode 10, the ADC converter, the DAC converter, the electronic system for measuring BI, microcontroller, the Bluetooth module, as shown in FIG. 4.

The electrodes 10 are metal plates against which the measured person puts hands. Putting a hand causes a flow of a sinusoidal current with a very low voltage through the body. The ADC converter is an electronic system converting an analog signal from the electrodes into a digital signal. The DAC converter is an electronic system converting a digital signal from the BI measurement system into a sinusoidal current.

The electronic BI measurement system 9 is a specialized system for measuring the bioelectrical impedance. It measures the voltage of the current flowing through the human body and on this basis defines BI. The digital signal from the electronic system for measuring BI is connected to one of the digital inputs of the microcontroller. On the basis of BI and data on age, sex, body weight and height, it calculates the percentage of fat content in the body weight. The Bluetooth module is used for communication of peripheral devices with the microcontroller in order to read the measurement performed.

The pulse rate measurement system 14 is an electronic device used to measure the pulse rate in the examined person and providing measurement data via Bluetooth. The pulse rate measurement system 14 consists of the following components: the diode sensor, the microcontroller and the Bluetooth module as shown in FIG. 5.

The pulse rate measurement system is put on the finger with the clip containing LEDs: red and infrared, as well as a tight sensor. Based on the analysis of the quantity of light emitted by oxygenated/deoxygenated hemoglobin, the light sensor converts the information on the change in the absorbed lighting into voltage changes at the output. The analog signal output of the sensor is connected to one of the analog inputs of the microcontroller. The microcontroller calculates the pulse rate based on changes in the signal at the analog input. The Bluetooth module is used for communication of peripheral devices with the microcontroller in order to read the measurement performed.

The arterial blood pressure measurement system 13 is a device for measuring arterial blood pressure providing measurement data via Bluetooth. The measurement system consists of the following components: the sleeve for an analog measurement of changes in blood pressure, the amplifier with the ADC converter, the microcontroller and the Bluetooth module as shown in FIG. 6.

The device comprises the “sleeve” into which the examined person puts their arm. When the sleeve is inflated to a suitable pressure, at the output of the converter, the information appears about changes in pressure caused by blood flow in the place where the converter is in contact with the skin in the form of voltage changes ranging from 0 to 40 mV. The amplifier with the ADC converter is used to convert the voltage received from the pressure converter to the range of 0-5 V. The altered signal is sent to the ADC (analog to digital) converter which converts the analog signal into a digital signal. The digital signal from the ADC convener is connected to one of the digital inputs of the microcontroller there. A digital measurement of pressure and number of heart heats per second is carried out in the microcontroller. The Bluetooth module is used for communication of peripheral devices with the microcontroller in order to read the measurement performed.

The waist and hip circumference measurement device 8 was designed as a component of the device in a child-friendly way to encourage children to measure the waist circumference. Accordingly, the measurement device is so positioned in the device as to resemble in its appearance e.g. a tongue of a giraffe.

The waist circumference measurement device 8 is based on “Digital Type Measure” devices which are in the form of:

a) a measuring tape in centimeters (to parts of a millimeter) and inch scale;

b) a measurement system which measures the level of projection of the measuring tape.

After closing the circuit of the measuring tape, the measurement system sends the measurement to the tablet, which presents the measurement on the screen for the given examination via the device.

The motion sensor system 4 is an electronic device for detecting movement within a radius of 7 meters from the device. The device consists of the following components: the motion sensor, the microcontroller, and the Bluetooth module.

The server 12 is the computer of the device collecting, processing and storing data collected from the devices. Another function is to provide an informational website, where users have access to their profiles with the data of their children.

The device software in the mobile application version presents a looped animation encouraging children In use the device. When using the device, the informational tablet 6 presents information suggesting step-by-step what to do to move through the process of measurements and data entry. Additionally, the user can receive information about healthy nutrition and the benefits resulting from regular screening in children.

The application is installed on the informational tablet 6. The function performed by the application is the presentation of two animations to encourage use of the device.

The first animation presents information about how the device works and provides information on the issues of healthy nutrition, the benefits resulting from regular measurement of BMI or BI. At the same time, it encourages the child to use the device (to carry out measurements). The second animation is triggered with the motion sensor. After launching, it provides instructions what to do and in what order to perform measurements with the device. The application is to lead step-by-step the parent/guardian/person supporting measurements through the entire process of measuring body weight, BI, waist circumference, as well as entering and storing data. The application also communicates with the microcontroller (via Bluetooth) coupled to the motion sensor in order to run animation encouraging the child to use the device.

The mobile application works in such a way that after starting the presentational application, the mobile application enters the mode of animation encouraging use of the device. The animation and the information about the device and the measurement process are presented alternately. When a person appears in the vicinity of the device, which is detected by the motion sensor—there starts the second animation, which instructs what to do and in what order to perform measurements with the device. After completion of the measurement procedure, the application checks whether there is someone nearby (checking the status of the motion sensor). If yes, then the animation is repeated. If not, it switches to the first animation presentation mode (with information encouraging to use of the device). In the event that the procedure of measurement is initiated, information showing step-by-step instruction of the measurement procedure is displayed in the informational application. After completion (or cancellation) of the measurement procedure, the application switches to the first animation display mode.

On the other hand, the second mobile application used to enter data on the tablet is used to log in/register, to enter the information relating to the child and to measure waist circumference. The mobile application for data entry is installed on the interactive tablet 7. Functions performed by the application include:

a) user registration—in order to send the measurement of the user and to access to own profile at the informational website they must register in the system providing data: e-mail address, access password;

b) user logging—if the user already has an account registered in the system they can log in it so that subsequent measurements are assigned to their account;

c) entering data about a person, for whom measurements are performed: name, age, sex, waist circumference measured;

d) information about the current status of the measurement process.

This application is to carry out the measurement process and data entry in an interactive way. By default, the application in the mode before the measurement process displays a welcome screen. The user touches the screen to launch the application in the interactive mode. At the start, the user is prompted to log in, and if they do not have an account, the user is prompted to register. In the event of registration, they are directed to the registration form, which they fill in by providing: e-mail address (being also the username), password (twice for confirmation), and name.

In the case of logging in, e-mail address as the user name and password are entered. After logging in a menu appears where the user sees a list of their children for whom they have ever performed a measurement. If they have never performed a measurement, this list is marked as empty. When the user wants to carry out another measurement, they select the child from the list. If they perform the measurement for that child for the first time, they add a new child providing their details: name, date of birth, sex. After selecting/entering the child they select the measurement start button. During the measurement one needs to follow the instructions displayed on the informational tablet. After carrying out the measurement by the devices, the application will prompt the user to perform the measurement of waist circumference with the measuring tape available on the device. After carrying out the measurement of waist circumference the measured value is entered in the application via the data input screen. In the extended version, the device will also be able to measure pulse rate and arterial blood pressure. These two measurements require a proper behavior of the measured person. When measuring the pulse, the finger must be put to the pulse sensor, and in the case of measuring the arterial blood pressure, the arm must be put into the sleeve of the measuring device. Each of these additional measurements is triggered upon user's request in the interactive tablet. The user confirms the readiness of the measured person for the measurement by selecting an appropriate button to start either the measurement of pulse rate or the arterial blood pressure. After performing the measurement procedure, there are messages displayed upon completion of the measurements and a summary of the measurements. The user will be able to repeat the measurement by selecting the “Repeat measurement” button. To the e-mail address of the user the information with the measurement results and the summary will be sent. In the event of an error occurring in the measurement procedure, the user can restart the measurement again by selecting the “Restart measurement” button. At any time, the user can cancel the measurement procedure. Then, the interactive tablet will switch into default mode in which it was before the measurement. The informational tablet will enter the mode displaying the first animation and the information about the device. In the case, where there is no interaction after the measurement procedure error occurs or after the measurement procedure is completed, the interactive application switches to the default mode (before the measurement) after a predetermined time (e.g. 1 minute).

Another application is the mobile application (on the tablet) to communicate with the microcontroller. This application is used to communicate with the microcontroller using the Bluetooth device. Its task is to control the operation of the microcontroller and reading the data collected by the microcontroller from the devices (such as weight, BI measurement, and height measurement).

The communication software in the device is in the form of a mobile application running in service mode (in the background) on the interactive tablet 7.

This application performs the following functions:

a) control over the body weight measurement process;

b) reading the last body weight measurement;

c) control over the height measurement process;

d) reading the last height measurement;

e) control over the BI measurement process;

f) reading the last BI measurement (more precisely, reading the computed percentage value of body fat share in total body weight);

g) control over the pulse rate measurement process;

h) reacting the last pulse rate measurement;

i) control over the arterial blood pressure measurement process;

j) reading the last arterial blood pressure measurement;

k) storing the read data and linking them to the user profile;

l) sending the measurement data to the sender.

The communication application is to control the measurement process in the subsequent measuring devices and reading measurement data from the devices. Communication takes place via Bluetooth. By default, the communication application is in standby mode waiting for the message from the interactive tablet about starting the measurement.

At the start of the measurement procedure, the communication application starts controlling the first measuring device, which is the height measurement system. After sending a request to the height measurement system, reading of the measurement data is started. The use of the ultrasonic device also allows measuring the length and corpulence of the foot.

When the read data are correct, it stores them and starts the body weight measurement. It sends a request to the body weight measurement system and reads the information on the measurement carried out.

In the case of correct data, it stores them and starts the BI (fat tissue) measurement procedure. It sends a request to start the measurement to the BI measurement system and then starts reading the measurement data. In the case of correct data reading, it stores them and enters the standby mode. In the extended version the device will also be able to measure pulse rate and arterial blood pressure. These two measurements require a proper behavior of the measured person. When measuring the pulse, the finger must he held against the pulse sensor, and in the case of measuring the arterial blood pressure, the arm must be put into the sleeve of the measuring device. Each of these additional measurements is triggered upon user's request in the interactive tablet. The user confirms the readiness of the measured person for the measurement by selecting an appropriate button to start either the measurement of pulse rate or the arterial blood pressure. Upon confirmation of the pulse rate measurement, the communication application sends a request to the pulse rate measurement system and then starts reading the measurement data. In the case of reading correct data, it stores them. In the case of the arterial blood pressure measurement, its start in the interactive tablet causes sending a request for starting the measurement via the communication application to the arterial blood pressure measurement system. After a set (configurable) delay time, the communication application starts reading the data. In the case of reading correct data, it stores them. After successful completion of the measurement procedure, the communication application sends the stored data to the interactive application and sends them to the server. In the case of a measurement error, it sends an error message to the interactive application.

Software in each of the microcontrollers in the measurement systems largely coincides one with another, regardless of which measurement system it is in. The microcontroller reads via Bluetooth the request to perform the measurement with the data for calculation (if needed). Depending on the measuring device, it sends to the device a signal to initiate the measurement process (e.g. pulse rate measurement) or reads the measured values directly from the device. The microcontroller calculates the measurement as the measured value is not necessarily the expected value, e.g. in the BI measurement, one needs to use the value of the measured voltage and the data on age, sex or body weight. The calculated value is stored by the microcontroller until the next measurement request. Software of the microcontroller provides the calculated measured values via Bluetooth as well as any measurement statuses like: measurement in progress, measurement error, measurement completed or other.

In the case of the software of the motion sensor microcontroller, the course of action involves the software that on an ongoing basis reads data from the motion sensor and in the case of the sensor detecting people near the device it provides information about this via Bluetooth. Otherwise, it provides via Bluetooth, the information about the lack of movement in the vicinity of the device.

The mobile application (on the interactive tablet 7) for sending data to the server is to collect data entered by the user and measurements collected from the devices and to send them to the server using the http protocol.

The applications managing devices connected 10 the microcontrollers read the measurement of height bioelectrical impedance, body weight, as well as pulse rate and arterial blood pressure in the extended version. The additional important function of the application is to provide the measurement data to other devices via Bluetooth. The application of the motion sensor microcontroller reads the information from the motion sensor and provides it outside using Bluetooth.

For the BI measurement there is a built-in application running on the microcontroller connected to the system TX AFE4300 measuring the value of BI. The task of the application is to manage the process of measuring BI to calculate the percentage value of body fat, to store and provide this value to be read by external devices using Bluetooth.

For the body weight measurement there is a built-in application running on the microcontroller coupled with a scale that allows control of the body weight measurement process, as well as to store and provide the data of the last measurement for external devices via Bluetooth.

For the pulse rate measurement there is a built-in application running on a microcontroller that allows control of the pulse rate measurement process, as well as to store and provide the measurement data for external devices using Bluetooth.

For the arterial blood pressure measurement there is a built-in application running on the microcontroller that allows control of the arterial blood pressure measurement process, as well as to store and provide the measurement data outside via Bluetooth.

On the server of the applications software has been installed to receive, process and store the data sent from the devices.

The functions performed by the server software are:

a) receiving the user's registration data sent, setting up a user profile, sending a confirmation e-mail about setting up a new account;

b) user authentication;

c) receiving the measurements data sent for N children associated with a particular user profile;

d) storage of measurement data in the database (assigning them to an appropriate user profile);

e) receiving diagnostic reports from the devices.

The application is awaking the data sent with the established http protocol. Upon receipt of the data, it analyzes the type of request and performs operations depending on its type. For user authentication, it verifies whether the given username and password (sent within the data) are correct. If so, it establishes a user's session and allows the user to operate in the web application. In the case of receiving a request for storing the measurement data, the application checks whether the data are correct and whether they relate to a user existing in the system. If so, it stores the transferred data in the database by assigning them to a particular user.

In the case of receiving by the application a diagnostic report, the application checks whether the data are correct and whether they relate to a device existing in the system. If so, it stores the transferred report in the database by assigning it to a particular device.

If the user has sent a registration form, the application checks its validity. In the case of errors of filling out the form, the application returns to the interactive application an error code together with its description that will Ile displayed to the user.

Specification of the informational website (www which is made in the RWD technology—a responsive website adjusted to web and mobile browsers).

The informational website is both a place to present information related to healthy nutrition, he need for screening measurements, as well as an application, where every user has access to their profile and associated set of measurements.

The functions performed by the website:

a) presentation of content (articles, news, etc.);

b) logging the user to their profile;

c) registration of a new user;

d) presentation of given information on measurements for a selected child.

The user enters the informational website. In order to log into own profile, they select the log-in button. They enter their username and password and, in the case of correct authentication, get access to their profile. In the case that the user does not have an account, they can register on the website. To do so, they click the “Register” button and fill out the registration form. Once the registration is complete, an activation link is sent to the provided e-mail address of the user. After opening it, the user confirms their registration on the website.

The user has on their profile a list of people currently using the device. In order to obtain information about a person they click it. They receive a screen where they can select the tab for:

a) measurements;

b) diagrams;

c) tips;

d) contact with a physician.

After selecting the measurements tab they have access to a list of all the measurements for the person.

After selecting a particular measurement designated with a dale the) receive the raw data on the performed measurements of:

a) height;

b) weight;

c) waist circumference;

d) BI;

e) pulse rate;

f) arterial blood pressure.

Selecting the “diagrams” tab allows to view diagrams of every measurement over time. The user specifics the start and end dates, as well as the measured values that they want to appear on the graph. The generated diagram contains a series of diagrams of measured values which allows their better analysis. The user can export every diagram to pdf format. Entering the “tips” tab causes the display of an information report containing interpretations of the measurement results, including growth charts with the information on which value corresponds to a given measurement and a set of tips on healthy nutrition and conducting healthy lifestyles.

The “contact with a physician” tab allows sending complete information in the form of measurements and graphs to predefined physicians to their e-mail addresses.

The mobile application installed on the interactive tablet checks the correctness of operation of individual components of the device and sends to the server information about the problems in the operation.

In the event of the most common problem, which is a temporary power outage on the screen of the tablet (tablets have batteries so they will operate for a few hours without external power supply), a message on a failure of the entire device is displayed. After restoring power, the message disappears.

The self-diagnostics application is installed on the interactive tablet (for data entry) and operated in the service mode (in the background). The functions performed by the application are:

a) cyclic diagnostics of the correctness of operation of the microcontroller connected to the measuring devices;

b) cyclic diagnostics of the measuring devices execution of test measurements when the device is not in use (once a day);

c) cyclic diagnostics of the operation of the informational tablet;

d) sending diagnostic reports to the server.

The self-diagnostics application is used to check the performance of individual components of the device. The application will cyclically carry out tests of the entire system and gather information about operational errors during the measuring process. Upon starting, the self-diagnostics application connects to the presentational tablet a network and attempts to communicate with it. In the case of communication failure, it stores the error message. Then, it proceeds to the inspection of the measurement systems. It attempts to communicate via Bluetooth with each measurement system.

In the case of failure to communicate with any measurement system, it stores this as an operational error. It also attempts to communicate with the motion sensor. If the device does not respond, an operational error is stored. After the inspection it sends to the server a report on the test carried out, sending information about occurring errors.

In the event that an error of Bluetooth communication with any measurement system or with the presentational tablet occurs during the measurement process, an error report is created and sent to the server.

The admin panel application includes software for managing informational website and diagnostics of the devices. It manages the content of the informational website and allows the presentation of information about the operation of the devices.

The admin panel web application is installed on the server. This application performs the following functions:

a) authentication of the administrator;

b) overview of diagnostic reports sent cyclically by the devices;

c) overview of the devices and their configurations;

d) presentation of the diagnostic report.

The administrator can enter the admin panel website page, where they enter username and password. After logging in correctly, they get access to the application. In the application they have access to tabs with a list of devices and with diagnostic reports. In order to view the configuration of a given device they click the record of the given device in the list. They receive the login with the configuration information. Additionally, next to the device there is the information on whether it operates properly, or whether an operational error occurred. In order to review diagnostic reports, the administrator selects the tab for diagnostic reports. In a report, there is a list of devices with summary lists of sent diagnostic reports in the form of a table linked to each device. The administrator selects from the list of reports the given report to view its details. In the details of the diagnostic report, they find the information about the diagnostic tests performed and their result—an error or correct operation of the device.

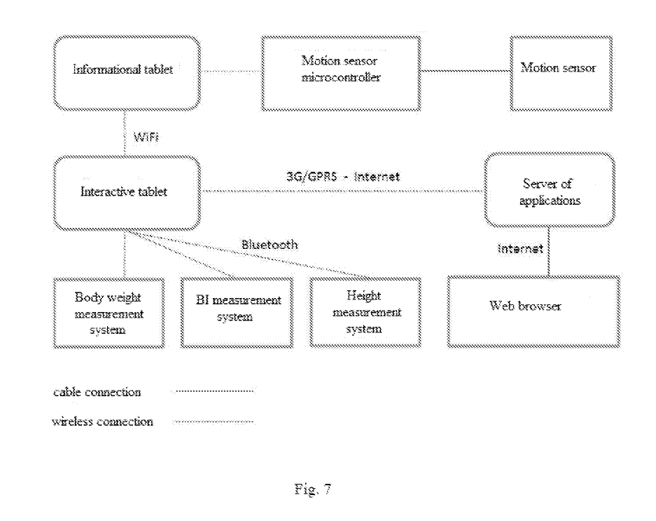

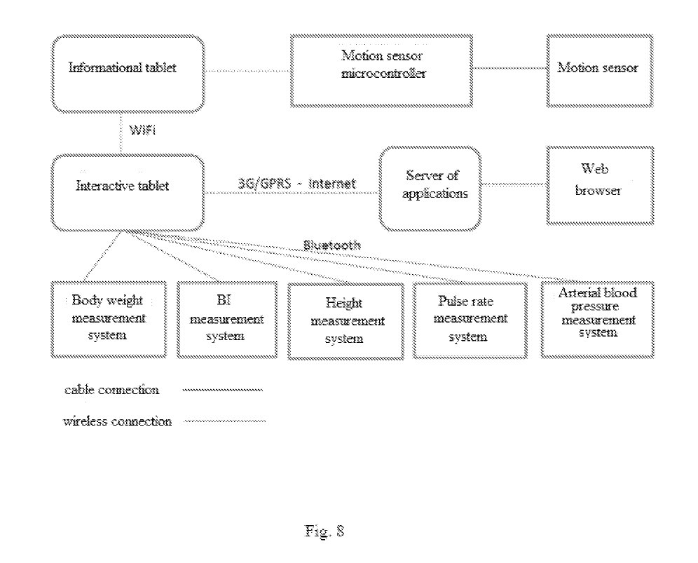

The architecture of the device being the object of the invention is presented below and in FIG. 7, including:

a) basic version;

b) extended version;

c) diagrams of particular measurement systems.

The architecture of the device comprises the informational tablet 6 communicating with the motion sensor microcontroller 4 using the Bluetooth device. It controls the microcontroller and reads data from it using serial transmission. The motion sensor microcontroller is connected through wires to the motion sensor, directly reading its current state from the motion sensor. The informational tablet 6 is connected to the interactive tablet 7 using Wi-Fi in order to exchange the information on the measurement process and the diagnostic information. The exchange of information will be carried nut using the HTTP protocol. The interactive tablet 7 connects to the microcontroller via the Bluetooth device. Communication with the microcontrollers (control/readout of data) is carried out using serial transmission. The body weight measurement system 11, the BI measurement system 9, the pulse rate measurement system 14, the arterial blood pressure measurement system 13, and the height measurement system 5 are connected to the sensors or to the measuring system with wires. The interactive tablet 7 is connected to the Internet using the built-in 3G modem. Using an SSL secure transmission, it communicates with the server in order to transfer measurement data and diagnostic data. The application server 12 is connected to the Internet via a broadband connection. The user, using a web browser, enters the informational website served from the server of applications.

1. A measuring device for comprehensive screening for children and adolescents, said measuring device comprising:
a body comprised of a vertical component having a top and a bottom;
an arm mounted at said top of said vertical component and forming an angle of 90°, said arm rotationally in the a horizontal plane;
a base positioned on said bottom of said vertical component;
a motion sensor system and a height measurement system on said arm; and,
at least one interactive electronic device, a bioelectrical impedance (BI) measurement system, and a waist and hip measurement system on said component,
wherein said base comprises a built-in body weight measurement system.

2. The measuring device according to claim 1, further comprising: a motion sensor system being comprised of a motion sensor, a microcontroller and a Bluetooth module.

3. The measuring device according to claim 1, wherein said height measurement system is comprised of an ultrasonic distance sensor, a microcontroller and a Bluetooth module.

4. The measuring device according to claim 1, wherein the interactive electronic device is comprised of an interactive tablet with a built-in communication modem.

5. The measuring device according to claim 1, wherein said bioelectrical impedance (BI) measurement system is comprised of an electrode, an ADC converter, a DAC converter, an electronic system for the BI measurement, a microcontroller and a Bluetooth module.

6. The measuring device according to claim 1, wherein said waist and hip circumference measurement device is comprised of a hoop or a belt or a measuring tape with a centimeter and inch scale, as well as the system measuring the level of projection of the measuring tape.

7. The measuring device according to claim 1, wherein said body weight measurement system is comprised of a pressure sensor load, an ADC converter, a microcontroller and a Bluetooth module.

8. The measuring device according to claim 1, further comprising: an interactive electronic device being comprised of an informational tablet.

9. The measuring device according to claim 1, further comprising: a pulse rate measurement system being comprised of a diode sensor, a microcontroller and a Bluetooth module.

10. The measuring device according to claim 1, further comprising: an arterial blood pressure measurement system, being comprised of a sleeve for analog measurement of changes in blood pressure, an amplifier with an ADC converter, a microcontroller and a Bluetooth module.

11. The measuring device according to claim 1, further comprising:
software.

12. The measuring device according to claim 1, further comprising: a Bluetooth module for communication of peripheral devices with a microcontroller in order to read the measurement carried out.

13. The measuring device according to claim 1, further comprising: a server being comprised of a computer collecting, processing and storing the collected data.

14. A measurement system for comprehensive screening for children and adolescents, comprising:
a measuring device, according to claim 1; and
a server connected to said measuring device via a communication network, wherein said measuring device performs measurement of a given parameter after initiation by a user, stores a result and sends the result to said server where results are saved, processed and stored.

15. The system according to claim 14, wherein said server is comprised of a communication module.
